SEQUENCE LISTING (14 sequences)

SEQ ID NO: 1 - protein, 121 aa, Artificial Sequence
  Synthetic
EVQLVESGGGVVQPGRSLRLSCAASGFTFSSYLMSWVRQAPGKGLEWVATISGGGGDTYFPDSVKGRFTISRDNSKNTLYLQMNSLRAEDTAVYYCVRFGGAGYYWYFDVWGQGTLVTVSS

SEQ ID NO: 2 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 111 aa, Artificial Sequence
  Synthetic
EIVLTQSPATLSLSPGERATLSCRASKSVDDSGISFMHWYQQKPGQAPRLLIYAASNQGSGIPARFSGSGSGTDFTLTISSLEPEDFAVYYCHQTKEVPWTFGQGTKVEIK

SEQ ID NO: 4 - protein, 107 aa, Artificial Sequence [sequence omitted]

SEQ ID NO: 5 - DNA, 31 bp, Artificial Sequence
  Primer
gaagatctaatatttttattgcaactccctg

SEQ ID NO: 6 - DNA, 31 bp, Artificial Sequence
  Primer
ctagctagcttataaaaggcgcgtcccgtgg

SEQ ID NO: 7 - DNA, 25 bp, Artificial Sequence
  Primer
gctctagattgcgacgccccggctc

SEQ ID NO: 8 - DNA, 35 bp, Artificial Sequence
  Primer
ccttaattaaggttaccaccctgtagccccgatgt

SEQ ID NO: 9 - DNA, 31 bp, Artificial Sequence
  Primer
tcccatggatttaacaaacgggggggtgtcg

SEQ ID NO: 10 - DNA, 28 bp, Artificial Sequence
  Primer
ggcccccgaggccagcatgacgttatct

SEQ ID NO: 11 - DNA, 32 bp, Artificial Sequence
  Primer
gagtaaccgccccccccccatgccaccctcac

SEQ ID NO: 12 - DNA, 31 bp, Artificial Sequence
  Primer
gtgttttactgccactacacccccggggaac

SEQ ID NO: 13 - protein, 121 aa, Artificial Sequence
  Synthetic
EVMLVESGGGLVKPGGSLKLSCAASGFTFSSYLMSWVRQTPEKRLEWVATISGGGGDTYFPDSVKGRFTISRDNVKNNLYLQMSSLRSEDTALYYCVRFGGAGYYWYFDVWGAGTTVTVSS

SEQ ID NO: 14 - protein, 111 aa, Artificial Sequence
  Synthetic
DIVLTQSPASLAVSLGQRATISCRASKSVDDSGISFMHWFQQKPGQPPKLLIYAASNQGSGVPARFRGSGSGTDFSLNIHPMEEDDTAMYFCHQTKEVPWTFGGGTKLEIK

(19) United States
(12) Patent Application Publication
(10) Pub. No.: US 2024/0024392 A1
(43) Pub. Date: Jan. 25, 2024

(54) ONCOLYTIC HERPES SIMPLEX TYPE 1 VIRUSES FOR TREATMENT OF BRAIN TUMORS

(71) Applicant: IMMVIRA CO., LIMITED, Shenzhen, Guangdong (CN)

(72) Inventors: Xiaoqing CHEN, Shenzhen (CN); Yuanyuan LIU, Shenzhen (CN); Grace Guoying ZHOU, Shenzhen (CN)

(21) Appl. No.: 18/265,248

(22) PCT Filed: Dec. 3, 2021
(86) PCT No.: PCT/CN2021/135336

§ 371 Date: Jun. 3, 2023

(30) Foreign Application Priority Data
Dec. 4, 2020 (WO) PCT/CN2020/133943

(51) Int. Cl. A61K 35/763; C07K 14/035; C12N 15/86; C07K 14/54; C07K 16/28; A61P 35/00

(52) U.S. Cl. CPC A61K 35/763; C07K 14/035; C12N 15/86; C07K 14/5434; C07K 16/2827; A61P 35/00

(57) ABSTRACT

Disclosed herein is an oncolytic HSV-1 virus genetically engineered for treatment of brain tumors, which lacks both copies of gamma 34.5 gene and an internal inverted repeat region and is optionally incorporated with immunostimulatory and/or immunotherapeutic genes. The oncolytic HSV-1 virus exhibited superior anti-tumor activity specifically in brain tumors. A pharmaceutical composition comprising the oncolytic HSV-1 virus and a pharmaceutically acceptable carrier, and a method of treatment of a brain tumor using the same is also disclosed.

### TECHNICAL FILED

The present disclosure relates to an oncolytic virus for treatment of tumors, and in particular, to an oncolytic Herpes Simplex Virus type 1 (oHSV-1) genetically engineered for treatment of brain tumors. The present disclosure also relates to a method for treating brain tumors using the recombinant oncolytic virus disclosed herein, and pharmaceutical compositions and uses thereof.

### BACKGROUND

Primary tumors of the brain can arise from different types of cells in the central nervous system. Medulloblastomas are derived from precursors of neuronal cells while astrocytomas are derived from the astrocytic subset of glial cells, and oligodendrogliomas are derived from the oligodendroglia precursor subset of glial cells. Other types of primary tumors are derived from cells that form the inner and outer linings of the brain such as ependymomas from ependymal cells, and meningiomas from cells that comprise the meninges, respectively. Glioblastoma multiforme (GBM) derived from astrocytes is the most common and deadliest primary brain tumor and is therefore classified as astrocytoma WHO Grade IV.

The current treatment regimen for malignant glioblastoma multiforme (GBM) is tumor-resection followed by chemo- and radiation therapies. Despite the proven safety of oncolytic herpes simplex virus (oHSV) in clinical trials for GBMs, its efficacy is sub-optimal mainly due to insufficient viral spread post-tumor resection. Glioblastoma multiforme (GBM) is the most common brain tumor in adults and despite great advances in its molecular understanding it remains one of the most difficult to treat malignancies. Although GBM tumor resection constitutes an important therapeutic intervention, standard treatment with radiation and temozolomide chemotherapy post-tumor resection only provides modest clinical benefits. Therefore, the development of novel local therapeutics that can be administered directly into the GBM tumor resection cavity post tumor debulking are urgently needed.

Previous studies attempting to use local therapy with clinically approved Gliadel wafers, polyanhydride wafers containing the chemotherapeutic agent, BCNU, in the cavity of resected GBM, have been shown to have limited therapeutic benefit. In the ongoing search for therapeutics that are capable of eliminating such tumor residues post tumor resection, oncolytic viruses have shown great potential in preclinical studies. These viruses are typically genetically engineered so as to only replicate in and kill neoplastic cells, an approach that fits well in the brain where actively proliferating tumor cells are amidst non- or slowly-proliferating normal cells. Among therapeutic viruses, oHSV is one of the most promising candidates for GBM therapy as it is an inherently neurotropic virus and is less dependent on certain host cell receptors, mutations or intracellular pathways for its oncolytic effect. Also, oHSV has a well-studied genome and a large transgene capacity for insertion of additional therapeutic genes to further enhance its oncolytic potency. Although phase I and Ib oHSV clinical trials conducted to date for GBM have shown signs of anti-tumor activity, clinical response rates have been sub-optimal.

### SUMMARY OF THE INVENTION

The inventors have surprisingly found that an oHSV-1 with deletions of both copies of γ34.5 gene and an inverted internal repeat region has unexpectedly superior antitumor activity specifically against brain tumors over non-brain tumors, when compared to existing oHSV-1 viruses.

In one aspect, provided herein is an oncolytic Herpes Simplex Virus type 1 (oHSV-1) comprising a modified genome, wherein the modification comprises (a) an alternation of a copy of γ34.5 gene that is in a terminal repeat of the genome, rendering that copy of γ34.5 gene incapable of expressing functional ICP34.5 protein, and (b) a deletion of an internal inverted repeat region of the genome, causing one copy of each of double-copy genes and one copy of duplicated non-coding sequences within the internal inverted repeat region deleted, wherein the double-copy genes comprise genes encoding ICP0, ICP4, ICP34.5, ORF P and ORF O, and wherein all single-copy genes in both U_{L} and U_{S} components of the genome are intact such that they are capable of expressing respective functional proteins.

In some embodiments, the alternation comprises a deletion of all or part of the coding or regulatory region of the copy of γ34.5 gene.

In some embodiments, the duplicated non-coding sequences include introns of ICP0, LAT domain and “a” sequence.

In some embodiments, the all single-copy genes in both U_{L} and U_{S} components include U_{L}1 to U_{L}56 genes in the U_{L} component and U_{S}1 to U_{S}12 genes in the U_{S} component.

In some embodiments, the oHSV-1 is selected from the group consisting of strains F, KOS, and 17. In some embodiments, the deletion of an internal inverted repeat region causes excision of nucleotide positions 117005 to 132096 in the genome of F strain.

In some embodiments, the oHSV-1 has a genome isomer of prototype (P) and the deletion of an internal inverted repeat region starts from the stop codon of the last gene (e.g. U_{L}56) in the U_{L} component to the promotor of the first gene (e.g. U_{S}1) in the U_{S} component.

In some embodiments, the oHSV-1 is incorporated with a heterologous nucleic acid sequence encoding an immunostimulatory and/or immunotherapeutic agent, wherein the incorporation does not interfere with the expression of native genes of the HSV-1 genome. In some embodiments, the oHSV-1 is incorporated with a heterologous nucleic acid sequence encoding an immunostimulatory agent and an immunotherapeutic agent.

In some embodiments, the immunostimulatory agent is selected from a group consisting of GM-CSF, IL-2, IL-12, IL-15, IL-24 and IL-27. In some embodiments, the immunostimulatory agent is IL-12.

In some embodiments, the immunotherapeutic agent is an anti-PD-1 agent, an anti-CTLA-4 agent or both. In some embodiments, the immunotherapeutic agent is an anti-PD-1 agent. In some embodiments, the anti-PD-1 agent includes an anti-PD-1 antibody or an antigen-binding fragment thereof, such as Fab, scFv, (scFv)₂, Fab′ or F(ab′)2. In some embodiments, the anti-CTLA-4 agent includes an anti-CTLA-4 antibody or an antigen-binding fragment thereof, such as Fab, scFv, (scFv)₂, Fab′ or F(ab′)2. In some embodiments, the anti-PD-1 antibody or the anti-CDLA-4 antibody includes an modified form of antibody including an antibody drug conjugate (ADC), bispecific antibody and nanobody (or VHH).

In some embodiments, the heterologous nucleic acid sequence is incorporated into the internal inverted repeat region and/or between U_{L}3 and U_{L}4 genes in the U_{L} component.

In some embodiments, the oHSV-1 is incorporated with a heterologous nucleic acid sequence encoding IL-12 and an anti-PD-1 agent. In some embodiments, the heterologous nucleic acid sequence encoding IL-12 is incorporated into the internal inverted repeat region and the heterologous nucleic acid sequence encoding the anti-PD-1 agent is incorporated between U_{L}3 and U_{L}4 genes in the U_{L} component.

In another aspect, provided is a pharmaceutical composition for treatment of a brain tumor, comprising an effective amount of any of the oHSV-1 disclosed herein and a pharmaceutically acceptable carrier. In some embodiments, the brain tumor is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

In another aspect, provided is use of any of the oHSV-1 disclosed herein in the manufacturing of a drug for treatment of a brain tumor. In some embodiments, the brain tumor is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

In another aspect, provided is use of any of the oHSV-1 disclosed herein for treatment of a brain tumor. In some embodiments, the brain tumor is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

In another aspect, provided is a method for treatment of a brain tumor in a subject, comprising administering to the subject a therapeutically effective amount of any of the oHSV-1 disclosed herein or any of the pharmaceutical composition disclosed herein.

In some embodiments, a second therapy is administered to the subject before, at the same time, or after the oHSV-1 disclosed herein or the pharmaceutical composition disclosed herein is administered. In some embodiments, the second therapy is chemotherapeutic, radiotherapeutic, immunotherapeutic and/or surgery intervention. In some embodiments, the subject is a human being. In some embodiments, the brain tumor is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

### BRIEF DESCRIPTION OF THE DRAWINGS

These and other aspects and advantages of the present disclosure are obvious from the following description described in detail with reference to the accompanied drawings, in which:

FIG. 1 shows the genome structures of oHSV-1 constructs T3011, C5252, C8282, C1212 and R3616.

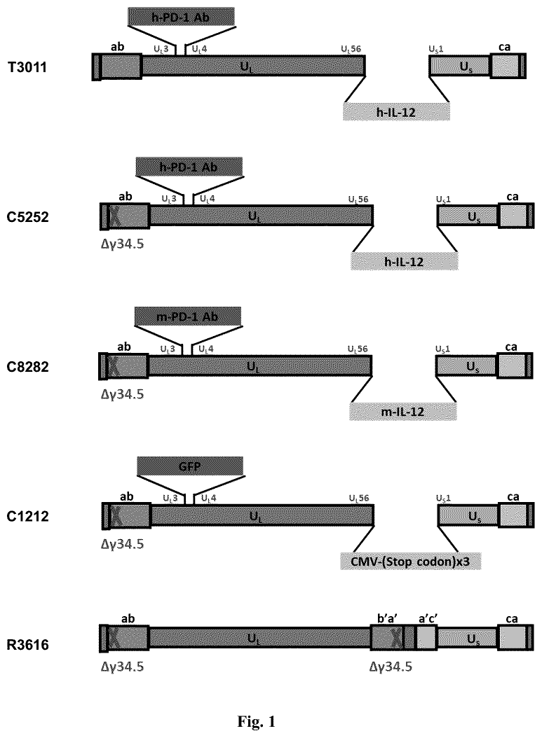

FIG. 2 shows the results of lacking ICP34.5 protein expression by C5252, C8282, and C1212. Six-well-plate of Vero cells were mock infected or infected at 1 PFU of HSV-1 (F), R3616, C5252, C8282, C1212 per cell. The cells were harvested at 6, 12 and 24 hours (H) post infection respectively. The ICP34.5 protein expression was detected by immunoblotting.

FIG. 3 shows the results of in vitro inhibitory assessment of C5252 on proliferation of human malignant glioma cells. There were 6 replicated wells for each sample, and these results were confirmed by another independent experiment. U87-MG, U138-MG, U373-MG, D54-MG and U251-MG were seeded onto 96-well plate (5000 cells/well) and infected with a series of titers of C5252/C1212 (0.01, 0.1, 1, 10, 33.33, 100 PFU/cell). After 48 hours infection, the inhibition rate of U87-MG, U373-MG, U138-MG, D54-MG and U251-MG were determined by Cell Titer-Glo Luminescent Cell Viability Assay and IC₅₀ were calculated.

FIG. 4 shows the results of the inhibitory effect of C5252 on normal cells and tumor cells. U373-MG, ACHN, HA and HRGEC were seeded onto 96-well plate (5000 cells/well) and infected with a series of titers of C5252 (0.01-500 PFU/cell). After 48 hours infection, the relative cell viability of U373-MG, ACHN, HA and HRGEC were determined by Cell Titer-Glo Luminescent Cell Viability Assay and IC₅₀ were calculated.

FIG. 5 shows the results of an efficacy study of C8282 in the treatment of GL261 subcutaneously implanted model in C57BL/6 mice. Thirty-two female C57BL/6J mice were subcutaneously inoculated with GL261 tumor cell (1×10⁶) at right flank. The mice were randomized into 4 groups of 8 mice in each group when tumor volume reached ˜70 mm³. Mice were intratumorally treated with C8282 (5×10⁴, 5×10⁵ or 5×10⁶ PFU/animal, 3 times in total, Q3d. Tumor volume and body weight were measured twice every week. Tumor volume and body weight was presented as mean±SEM.

FIG. 6 shows the results of an efficacy study of C5252 in the treatment of orthotropic U87 human glioma model in nude mice. Thirty female Balb/c nude mice were intracerebral inoculated with 5 μl of U87-Luc tumor cell at left striatum. The mice were randomized into 3 groups of 8 mice in each group 2 weeks after inoculation according luminescent signal in region of interest (ROI) acquired by IVIS image system. Mice were treated with C5252 (3×10⁴ or 3×10⁵ PFU/mouse in 5 μl) 6 times in total (D1, 4, 7, 10, 13, 16), every 3 days. IVIS was performed once weekly to monitor tumor growth.

### DETAILED DESCRIPTION

### Definitions

It is to be noted that the term “a” or “an” entity refers to one or more of that entity; for example, “an oncolytic HSV-1,” is understood to represent one or more oncolytic HSV-1 viruses. As such, the terms “a” (or “an”), “one or more,” and “at least one” can be used interchangeably herein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

As used herein, an “antibody” or “antigen-binding polypeptide” refers to a polypeptide or a polypeptide complex that specifically recognizes and binds to one or more antigens. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody also encompasses polypeptides or polypeptide complexes that, upon activation, possess antigen-binding capabilities.

The terms “antibody fragment” or “antigen-binding fragment”, as used herein, is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgGl, IgG2, IgG3, IgG4, IgAl and IgA2) or subclass of immunoglobulin molecule. For example, an anti-PD-1 antibody may refer to a Fab fragment or scFv thereof.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, “cancer” or “tumor” as used interchangeably herein is meant to a group of diseases which can be treated according to the disclosure and involve abnormal cell growth with the potential to invade or spread to other parts of the body. Not all tumors are cancerous; benign tumors do not spread to other parts of the body. Possible signs and symptoms include: a new lump, abnormal bleeding, a prolonged cough, unexplained weight loss, and a change in bowel movements among others. There are over 100 different known cancers that affect humans. The present disclosure is preferably applicable to solid tumors, more preferably to brain tumors.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an oHSV-1 or composition of the present disclosure used, e.g., for detection, for a diagnostic procedure and/or for treatment.

It will also be understood by one of ordinary skill in the art that modified genomes as disclosed herein may be modified such that they vary in nucleotide sequence from the modified polynucleotides from which they were derived. For example, a polynucleotide or a nucleotide sequence derived from a designated DNA sequence may be similar, e.g., have a certain percent identity to the starting sequence, e.g., it may be 60%, 70%, 75%, 80%, 85%, 90%, 95%, 98%, or 99% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Oncolytic Herpes Simplex Virus type 1

The HSV-1 genome consists of two covalently linked components, designated L and S. Each component consists of unique sequences (U_{L} for the L component, U_{S} for the S component) flanked by inverted repeats, i.e., terminal repeats and internal repeats. The inverted repeats of the L component are designated as ab and b′a′. The inverted repeats of the S component are designated as a′c′ and ca. Inverted repeats b′a′ and a′c′ constitute an internal inverted repeat region. The inverted repeats regions of both L and S components are known to contain two copies of five genes encoding proteins designated ICP0, ICP4, ICP34.5, ORF P and ORF O, respectively and large stretches of DNA that are transcribed but do not encode proteins including e.g., introns of ICP0, LAT domain, “a” sequences and etc.

Homologous recombination between the terminal repeats results in the inversion of the L and S components of HSV-1 genome, yielding four linear isomers at equimolar concentrations. The isomers are designated as P (prototype), I_{L} (inversion of the L component), I_{S} (inversion of the S component), and I_{SL} (inversion of both L and S components). HSV-1 genome encodes approximately 90 unique transcription units (genes), approximately half of which are essential for viral replication in a permissive tissue culture environment. The rest are dispensable for growth in cells in culture. However, these so-called ‘nonessential’ genes are most probably not dispensable for replication in animal system. They often encode functions that are involved in virus-host interactions, for example, inducing immune evasion and host cell shut-off.

Infected cell protein 34.5 (ICP34.5) is a protein encoded by the γ34.5 gene (also known as 7134.5), and it blocks a cellular stress response to viral infection. When a cell is infected by HSV, protein kinase R is activated by the virus' double-stranded RNA. Protein kinase R then phosphorylates a protein called eukaryotic initiation factor-2A (eIF-2A), which inactivates eIF-2A. EIF-2A is required for translation so by shutting down eIF-2A, the cell prevents the virus from hijacking its own protein-making machinery. Viruses in turn evolved ICP34.5 to defeat the defense; it activates protein phosphatase-1A which dephosphorylates eIF-2A, allowing translation to occur again. HSV lacking the γ34.5 gene will not be able to replicate in normal cells because it cannot make proteins. There are two copies of γ34.5 gene in the HSV-1 genome, flanking the U_{L} component, one at the terminal repeat and the other at the internal repeat.

In one aspect, the present disclosure provides an oncolytic herpes simplex virus type 1 (oHSV-1) genetically modified to render both copies of γ34.5 gene incapable of expressing functional ICP34.5 proteins, and the oHSV-1 is further modified to delete an internal inverted repeat region of the genome. The deletion of the internal inverted repeat region causes one copy of each of double-copy genes including genes encoding ICP0, ICP4, ICP34.5, ORF P and ORF O and one copy of duplicated non-coding sequences within the internal inverted repeat region deleted. However, all single-copy genes, including U_{L}1 to U_{L}56 and U_{S}1 to U_{S}12, in the U_{L} and U_{S} components of the genome are intact such that they are capable of expressing respective functional proteins.

In some embodiments, the modification comprises an alternation of a copy of γ34.5 gene that is in a terminal repeat of the genome, rendering that copy of γ34.5 gene incapable of expressing functional ICP34.5 protein. By “incapable of expressing functional ICP34.5 protein” it means γ34.5 is not detectable at protein or mRNA level in the engineered virus, or a ICP34.5 protein is expressed by the virus but is non-functional or partially functional. Measures for achieving the above is readily available in the art of genetic engineering and are known to a skilled person. For example, the alternation can comprise an insertion, a mutation, or an addition of one or more nucleotides in the coding or regulatory region of the copy of γ34.5 gene, or a deletion of all or part of the coding or regulatory region of the copy of γ34.5 gene. In some embodiments, the alternation comprises a deletion of all or part of the coding or regulatory region of the copy of γ34.5 gene.

The oHSV-1 as disclosed herein lacks both copies of γ34.5 gene. The other copy of γ34.5 gene, which is located within the internal repeat of the U_{L} component, is deleted through the deletion of the internal inverted repeat region of the genome. As described above, the internal inverted repeat region is consisted of an internal repeat of the U_{L} component and the internal repeat of the U_{S} component. One copy of double-copy genes including genes encoding ICP0, ICP4, ICP34.5, ORF P and ORF O and one copy of duplicated non-coding sequences are located within the internal inverted repeat region. Therefore, the deletion of the internal inverted repeat region will cause the deletion of the one copy of double-copy genes, including the other copy of γ34.5 gene, and the one copy of duplicated non-coding sequences. In some embodiments, the duplicated non-coding sequences include e.g., introns of ICP0, LAT domain, and “a” sequences. Therefore, in some embodiments, the deletion of the internal inverted repeat region of the genome results in deletion of one copy of each of ICP0, ICP4, ICP34.5, ORF P and ORF O and one copy of each of introns of ICP0, LAT domain, and “a” sequences. The other copy of each of ICP0, ICP4, ORF P and ORF O and the other copy of each of introns of ICP0, LAT domain, and “a” sequences is therefore preserved in the engineered oHSV-1 genome.

In the present disclosure, the deletion of the internal inverted repeat region is carried out in a precise manner to make sure that all single-copy genes, including U_{L}1 to U_{L}56 and U_{S}1 to U_{S}12, in the U_{L} and U_{S} components of the genome are intact such that they are capable of expressing respective functional proteins. In this context, “all single-copy genes in the U_{L} and U_{S} components of the genome are intact” is meant that the ORFs of each of these single-copy genes and regulating sequences necessary for expression of each ORF such as promoters and enhancers are intact, to ensure the expression of the ORFs are successful and the proteins translated from the ORFs are functional. By “intact” it means the coding sequences of each of the single-copy genes are at least functional but it does not mean the sequences have to be 100% percent identical to the naturally occurring sequences. The sequences may slightly vary in nucleotide sequence from naturally occurring sequences by including for example conservative substitutions or changes at “non-essential” regions. In this context, the sequences may be 90%, 95%, 98%, or 99% identical to the naturally occurring sequences.

Given that the positions of each of the single-copy gene in the HSV-1 genome is known in the art and depends on the strains and genome isomers of the HSV-1 virus, it will be appreciated by a skilled person in the art that the exact starting and ending positions of the nucleotides to be deleted in the internal inverted repeat region will vary from strains to strains and from isomers to isomers, but can be easily determined by known techniques in the art. It should be understood that the present disclosure is not intended to be limited to any specific genome isomers nor strains of an HSV-1 virus. In contrast, the present disclosure speculates all strains and isomers of the HSV-1 virus be useful.

For example, in an embodiment where HSV-1 F strain is used, the genome of which is available by GenBank Accession No. GU734771.1, the deletion of the internal inverted repeat region causes the excision of nucleotides 117005 to 132096 in the genome. It also will be appreciated by the person skilled in the art that other strains are also possible as long as the genome DNA is sequenced. Sequencing technologies are easily available in literature and on the market. For example, in another embodiment, the deletion may be performed on an HSV-1 strain 17, the genome of which is available by GenBank Accession No. NC_001806.2. In another embodiment, the deletion may be performed on a strain KOS 1.1, the genome of which is available by GenBank Accession No. KT899744.

In some embodiments, the deletion is precisely performed at predetermined positions such that an excision of a DNA fragment starting from the last gene in the L component (such as U_{L}56 in case of P isomer) to the first gene in the S component (such as U_{S}1 in case of P isomer) is achieved. Given the four different isomers existing for HSV-1 (i.e., isomers P, I_{S}, I_{L}, and I_{SL}), the names of the first genes and of the last genes will vary among isomers. In the context of the present disclosure, the numbering of the genes (i.e., the first and the last) in the U_{L} component is defined in the orientation from the terminal repeat of the U_{L} component to the internal repeat of the U_{L} component, and the numbering of the genes in the U_{S} component is defined in the orientation from the internal repeat of the U_{S} component to the terminal repeat of the U_{S} component. Therefore, in the case of isomer prototype (P), the first gene in the U_{L} component would be such as U_{L}1 gene and the last gene in the U_{L} component would be such as U_{L}56, and the first gene in the U_{S} component would be such as U_{S}1 gene and the last gene in the U_{S} component would be such as U_{S}12. In the case of isomer I_{S}, the first gene in the U_{L} component would be such as U_{L}1 gene and the last gene in the U_{L} component would be such as U_{L}56, and the first gene in the U_{S} component would be such as U_{S}12 gene and the last gene in the U_{S} component would be such as U_{S}1. In the case of isomer I_{L}, the first gene in the U_{L} component would be such as U_{L}56 gene and the last gene in the U_{L} component would be such as U_{L}1, and the first gene in the U_{S} component would be such as U_{S}1 gene and the last gene in the U_{S} component would be such as U_{S}12. In the case of isomer I_{SL}, the first gene in the U_{L} component would be such as U_{L}56 gene and the last gene in the U_{L} component would be such as U_{L}1, and the first gene in the U_{S} component would be such as U_{S}12 gene and the last gene in the U_{S} component would be such as U_{S}1.

The deletion of the internal inverted repeat region will not lead to a damage to the single-copy genes in the U_{S} or U_{L} component in such a way that the coding and regulatory sequences of the single-copy genes, including promoter sequences necessary for the expression of the single-copy genes, are intact. For example, in the case of isomer P, the deletion causes an excision of a DNA fragment starting from the end of the stop codon of such as U_{L}56 gene to the start of the promoter sequence of such as U_{S}1 gene. For example, in the case of isomer I_{L}, the deletion causes an excision of a DNA fragment starting from the start of the promoter sequence of such as U_{L}1 gene to the start of the promoter sequence of such as U_{S}1 gene.

The preservation of all single-copy genes and the other copy of each of ICP0, ICP4, ORF P and ORF O and the other copy of each of introns of ICP0, LAT domain, and “a” sequences in the engineered oHSV-1 genome provides a stronger virus, either before or after incorporation of inserted foreign genes. The oHSV-1 is therefore to the maximum extent resistant to environmental factors, such as temperatures, pressures, UV light, and etc. It also maximizes the range of cancer cells in which the oncolytic HSV-1 is effective.

Various genetic manipulation methods known in the art can be used to obtain the modified HSV-1 vector as described in the present disclosure. For example, bacterial artificial chromosomes (BAC) technology is used. As another example, COS plasmid can be used with the present disclosure. WO 2017/181420 disclosed an oHSV-1 vector constructed by BAC technology, the entire content of which is incorporated herein by reference.

The amount of foreign DNA sequences that can be inserted into the wild-type virus is limited because it interferes with the packaging of the DNA into virions. The precise deletion in the designated region provides an ideal space for insertion of foreign DNA sequences. According to an embodiment of the present disclosure, the deletion removes at least 15 k bp of the oncolytic virus vector such that a similar amount of foreign DNA sequences can accommodate. Other studies have shown that wild type genomes tolerate an additional 7 KB of DNA.

In some embodiments, the genetically engineered oHSV-1 is incorporated with a heterologous nucleic acid sequence encoding an immunostimulatory and/or immunotherapeutic agent. In the present disclosure, the incorporation of a heterologous nucleic acid sequence does not interfere with the expression of native genes of the HSV-1 genome such as any of the single-copy genes or the other double-copy genes as described above.

In some embodiments, the heterologous nucleic acid sequence is incorporated into the internal inverted repeat region. In some embodiments, the heterologous nucleic acid sequence is incorporated between adjacent single-copy genes in the U_{L} or U_{S} component. In some embodiments, the heterologous nucleic acid sequence is incorporated into the internal inverted repeat region and between adjacent single-copy genes in the U_{L} or U_{S} component. In some embodiments, the heterologous nucleic acid sequence is incorporated into the internal inverted repeat region and between U_{L}3 and U_{L}4 genes.

In some embodiments, the oHSV-1 comprises a heterologous nucleic acid sequence encoding an immunostimulatory agent. In some embodiments, the immunostimulatory agent is selected from a group consisting of GM-CSF, IL-2, IL-12, IL-15, IL-24 and IL-27. In an embodiment, the immunostimulatory agent is IL-12. In an embodiment, the immunostimulatory agent is a human or humanized IL-12.

In some embodiments, the oHSV-1 comprises a heterologous nucleic acid sequence encoding an immunotherapeutic agent. In some embodiments, the immunotherapeutic agent is selected from an anti-PD-1 agent, an anti-CTLA-4 agent or both. In an embodiment, the immunotherapeutic agent is an anti-PD-1 agent.

In some embodiments, the oHSV-1 comprises a heterologous nucleic acid sequence encoding both an immunostimulatory agent and an immunotherapeutic agent. In some embodiments, the immunostimulatory agent is selected from a group consisting of GM-CSF, IL-2, IL-12, IL-15, IL-24 and IL-27. In an embodiment, the immunostimulatory agent is IL-12. In an embodiment, the immunostimulatory agent is a human or humanized IL-12. In some embodiments, the immunotherapeutic agent is selected from an anti-PD-1 agent, an anti-CTLA-4 agent or both. In an embodiment, the immunotherapeutic agent is an anti-PD-1 agent.

In the embodiments where only one heterologous nucleic acid sequence encoding an immunostimulatory or immunotherapeutic agent is inserted, the heterologous nucleic acid sequence is preferably incorporated into the deleted internal inverted repeat region of the genome. In an embodiment, the heterologous nucleic acid sequence has a length similar to that of the deleted fragment. In an embodiment, the heterologous nucleic acid sequence has a length 20% longer or shorter than that of the deleted fragment. In another embodiment, the heterologous nucleic acid sequence has a length 15%, 10%, 5%, 4%, 3%, 2%, or 1% longer or shorter than that of the deleted fragment.

In an embodiment, the heterologous nucleic acid sequence has a length of less than about 18 k bp, about 17 k bp, or about 16 k bp. In an embodiment, the heterologous nucleic acid sequence has a length of more than about 10 k bp, 11 k bp, 12 k bp, 13 k bp, or 14 k bp. In an embodiment, the heterologous nucleic acid sequence has a length between about 14 k bp and about 16 k bp. In an embodiment, the heterologous nucleic acid sequence has a length of about 15 k bp.

In some embodiments, the oHSV-1 comprises at least two heterologous nucleic acid sequences encoding immunostimulatory and/or immunotherapeutic agents. In some embodiments, the oHSV-1 comprises heterologous nucleic acid sequences encoding two different immunostimulatory agents. For example, in one embodiment, the oHSV-1 comprises heterologous nucleic acid sequences encoding both IL-12 and GM-CSF. In another embodiment, the oHSV-1 comprises heterologous nucleic acid sequences encoding both IL-15 and GM-CSF. In a further embodiment, the oHSV-1 comprises heterologous nucleic acid sequences encoding both IL-12 and IL-15.

In some embodiments, the oHSV-1 comprises heterologous nucleic acid sequences encoding two different immunotherapeutic agents. In one embodiment, for example, the oHSV-1 comprises heterologous nucleic acid sequences encoding both an anti-PD-1 agent and an anti-CTLA-4 agent.

In the embodiments where more than one heterologous nucleic acid sequences encoding immunostimulatory and/or immunotherapeutic agents are incorporated, a first heterologous nucleic acid sequence is preferably inserted into the deleted internal repeat region of the genome. A second or further heterologous nucleic acid sequences may be inserted into the L component of the genome. In an embodiment, a second heterologous nucleic acid sequence is inserted between the U_{L}3 and U_{L}4 genes of the L component. In an embodiment, a second heterologous nucleic acid sequence is inserted between the U_{L}37 and U_{L}38 genes of the L component.

In an embodiment, the first heterologous nucleic acid sequence encodes IL-12 inserted into the deleted internal repeat region of the genome. In an embodiment, the second heterologous nucleic acid sequence encodes an anti-PD-1 agent inserted between the U_{L}3 and U_{L}4 genes of the L component.

It will be appreciated that the insertions of the one or more heterologous nucleic acid sequences into the oncolytic HSV-1 genome do not interfere the expression of native HSV-1 genes and the heterologous nucleic acid sequences are stably incorporated into the modified HSV-1 genome such that functional expressions of the heterologous nucleic acid sequences can be expected.

The heterologous nucleic acid sequences encoding the immunostimulatory and/or immunotherapeutic agents contain nucleic acid encoding a peptide or protein along with regulatory elements for the expression. Generally, the regulatory elements that are present in a recombinant gene and selected on the basis of the host cells to be used for expression that is operably-linked to the nucleic acid sequence to be expressed include a transcriptional promoter, a ribosome binding site, and a terminator. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the virus is introduced into the host cell). The term “regulatory sequence” is intended to include promoters, enhancers and other expression control elements (e.g., polyadenylation signals). Regulatory sequences include those that direct constitutive expression of a nucleotide sequence in many types of host cell and those that direct expression of the nucleotide sequence only in certain host cells (e.g., tissue-specific regulatory sequences).

Appropriate regulatory elements can be selected by those of ordinary skill in the art based on, for example, the desired tissue-specificity and level of expression. For example, a cell-type specific or tumor-specific promoter can be used to limit expression of a gene product to a specific cell type. In addition to using tissue-specific promoters, local administration of the viruses can result in localized expression and effect. Examples of non-tissue specific promoters that can be used include the early Cytomegalovirus (CMV) promoter (U.S. Pat. No. 4,168,062) and the Rous Sarcoma Virus promoter. Also, HSV promoters, such as HSV-1 IE promoters, can be used.

Examples of tissue-specific promoters that can be used in the technology include, for example, the prostate-specific antigen (PSA) promoter, which is specific for cells of the prostate; the desmin promoter, which is specific for muscle cells; the enolase promoter, which is specific for neurons; the beta-globin promoter, which is specific for erythroid cells; the tau-globin promoter, which is also specific for erythroid cells; the growth hormone promoter, which is specific for pituitary cells; the insulin promoter, which is specific for pancreatic beta cells; the glial fibrillary acidic protein promoter, which is specific for astrocytes; the tyrosine hydroxylase promoter, which is specific for catecholaminergic neurons; the amyloid precursor protein promoter, which is specific for neurons; the dopamine beta-hydroxylase promoter, which is specific for noradrenergic and adrenergic neurons; the tryptophan hydroxylase promoter, which is specific for serotonin/pineal gland cells; the choline acetyltransferase promoter, which is specific for cholinergic neurons; the aromatic L-amino acid decarboxylase (AADC) promoter, which is specific for catecholaminergic/5-HT/D-type cells; the proenkephalin promoter, which is specific for neuronal/spermatogenic epididymal cells; the reg (pancreatic stone protein) promoter, which is specific for colon and rectal tumors, and pancreas and kidney cells; and the parathyroid hormone-related peptide (PTHrP) promoter, which is specific for liver and cecum tumors, and neurilemoma, kidney, pancreas, and adrenal cells.

Examples of promoters that function specifically in tumor cells include the stromelysin 3 promoter, which is specific for breast cancer cells; the surfactant protein A promoter, which is specific for non-small cell lung cancer cells; the secretory leukoprotease inhibitor (SLPI) promoter, which is specific for SLPI-expressing carcinomas; the tyrosinase promoter, which is specific for melanoma cells; the stress inducible grp78/BiP promoter, which is specific for fibrosarcoma/tumorigenic cells; the AP2 adipose enhancer, which is specific for adipocytes; the a-1 antitrypsin transthyretin promoter, which is specific for hepatocytes; the interleukin-10 promoter, which is specific for glioblastoma multiform cells; the c-erbB-2 promoter, which is specific for pancreatic, breast, gastric, ovarian, and non-small cell lung cells; the a-B-crystallin/heat shock protein 27 promoter, which is specific for brain tumor cells; the basic fibroblast growth factor promoter, which is specific for glioma and meningioma cells; the epidermal growth factor receptor promoter, which is specific for squamous cell carcinoma, glioma, and breast tumor cells; the mucin-like glycoprotein (DF3, MUC1) promoter, which is specific for breast carcinoma cells; the mtsl promoter, which is specific for metastatic tumors; the NSE promoter, which is specific for small-cell lung cancer cells; the somatostatin receptor promoter, which is specific for small cell lung cancer cells; the c-erbB-3 and c-erbB-2 promoters, which are specific for breast cancer cells; the c-erbB4 promoter, which is specific for breast and gastric cancer; the thyroglobulin promoter, which is specific for thyroid carcinoma cells; the ofetoprotein (AFP) promoter, which is specific for hepatoma cells; the villin promoter, which is specific for gastric cancer cells; and the albumin promoter, which is specific for hepatoma cells. In another embodiment, the TERT promoter or survivin promoter are used.

For example, in some embodiments, heterologous nucleic acid sequences are operably linked to a promoter, for example, a CMV promoter or an Egr-1 promoter. In an embodiment, a nucleotide sequence encoding IL-12 is operably linked to an Egr-1 promoter. In another embodiment, a nucleotide sequence encoding a scFv-anti-hPD1 is operably linked to a CMV promoter.

In certain embodiments, the oHSV-1 of the present disclosure encodes one or more immunostimulatory agents (also called immune stimulating molecules), including cytokines such as IL-2, IL4, IL-12, GM-CSF, IFNγ, chemokines such as MIP-1, MCP-1, IL-8 and growth factor.

Alternatively, or in addition, the oHSV-1 of the present disclosure encodes one or more immunotherapeutic agents, for example a PD-1 binding agent (or anti-PD-1 agent), or a CTLA-4 binding agent (or anti-CTLA-4 agent), including antibodies or fragments thereof, for example an anti-PD1 antibody specifically binding to PD-1 or an anti-CTLA-4 antibody specifically binding to CTLA-4. The anti-PD-1 antibody may be a single chain antibody that antagonizes the activity of PD-1. In other embodiments, the oncolytic virus expresses an agent that antagonizes the binding of the PD-1 ligands to the receptor, e.g., anti-PD-L1 and/or PD-L2 antibodies, PD-L1 and/or PD-L2 decoys, or a soluble PD-1 receptor.

The PD-1 signaling pathway plays an important role in tumor-associated immune dysfunction. Infection and lysis of the tumor cells can invoke a highly specific antitumor immune response which kills cells of the inoculated tumor, as well as cells of distant, established, non-inoculated tumors. Tumors and their microenvironments have developed mechanisms to evade, suppress and inactivate the natural anti-tumor immune response. For example, tumors may down-regulate targeted receptors, encase themselves in a fibrous extracellular stromal matrix or up-regulate host receptors or ligands involved in the activation or recruitment of regulatory immune cells. Natural and/or adaptive T regulatory cells (Tregs) have been implicated in tumor-mediated immune suppression. Without wishing to be limited by theory, PD-1 blockade may inhibit Treg activity and improve the efficacy of tumor-reactive CTLs. Further aspects of the technology will be described in further detail below. PD-1 blockade may also stimulate the anti-tumor immune response by blocking the inactivation of T-cells (CTLs and helper) and B-cells.

In one aspect, the present technology provides an oncolytic virus that carries a gene encoding a PD-1 binding agent. Programmed Cell Death 1 (PD-1) is a 50-55 kDa type I transmembrane receptor originally identified by subtractive hybridization of a mouse T cell line undergoing apoptosis. A member of the CD28 gene family, PD-1 is expressed on activated T, B, and myeloid lineage cells. Human and murine PD-1 share about 60% amino acid identity with conservation of four potential N-glycosylation sites and residues that define the Ig-V domain. Two ligands for PD-1 have been identified, PD ligand 1 (PD-L1) and ligand 2 (PD-L2); both belong to the B7 superfamily. PD-L1 is expressed on many cell types, including T, B, endothelial and epithelial cells, and antigen presenting cells. In contrast, PD-L2 is narrowly expressed on professional antigen presenting cells, such as dendritic cells and macrophages.

PD-1 negatively modulates T cell activation, and this inhibitory function is linked to an immunoreceptor tyrosine-based inhibitory motif (ITIM) of its cytoplasmic domain. Disruption of this inhibitory function of PD-1 can lead to autoimmunity. The reverse scenario can also be deleterious. Sustained negative signals by PD-1 have been implicated in T cell dysfunctions in many pathologic situations, such as tumor immune evasion and chronic viral infections.

Host anti-tumor immunity is mainly affected by tumor-infiltrating lymphocytes (TILs). Multiple lines of evidence have indicated that TILs are subject to PD-1 inhibitory regulation. First, PD-L1 expression is confirmed in many human and mouse tumor lines and the expression can be further upregulated by IFN-7 in vitro. Second, expression of PD-L1 by tumor cells has been directly associated with their resistance to lysis by anti-tumor T cells in vitro. Third, PD-1 knockout mice are resistant to tumor challenge and T cells from PD-1 knockout mice are highly effective in tumor rejection when adoptively transferred to tumor-bearing mice. Fourth, blocking PD-1 inhibitory signals by a monoclonal antibody can potentiate host anti-tumor immunity in mice. Fifth, high degrees of PD-L1 expression in tumors (detected by immunohistochemical staining) are associated with poor prognosis for many human cancer types.

Oncolytic virotherapy is an effective method to shape the host immune system by expanding T or B cell populations specific for tumor-specific antigens that are released following oncolysis. The immunogenicity of the tumor-specific antigens is largely dependent on the affinity of host immune receptors (B-cell receptors or T-cell receptors) to antigenic epitopes and the host tolerance threshold. High affinity interactions will drive host immune cells through multiple rounds of proliferation and differentiation to become long-lasting memory cells. The host tolerance mechanisms will counterbalance such proliferation and expansion in order to minimize potential tissue damage resulting from local immune activation. PD-1 inhibitory signals are part of such host tolerance mechanisms, supported by following lines of evidence. First, PD-1 expression is elevated in actively proliferating T cells, especially those with terminal differentiated phenotypes, i.e., effector phenotypes. Effector cells are often associated with potent cytotoxic function and cytokine production. Second, PD-L1 is important to maintain peripheral tolerance and to limit overly active T cells locally. Therefore, PD-1 inhibition using a PD-1 binding agent expressed in the tumor microenvironment can be an effective strategy to increase the activity of TIL and stimulate an effective and durable anti-tumor immune response.

In one aspect, the present technology provides an oncolytic virus comprising a heterologous nucleic acid encoding an anti-PD-1 agent. In some embodiments, the anti-PD-1 agents contain an antibody variable region providing for specific binding to a PD-1 epitope. The antibody variable region can be present in, for example, a complete antibody, an antibody fragment, and a recombinant derivative of an antibody or antibody fragment. The term “antibody” describes an immunoglobulin, whether natural or partly or wholly synthetically produced. Thus, anti-PD-1 agents of the present technology include any polypeptide or protein having a binding domain which is specific for binding to a PD-1 epitope.

Different classes of antibodies have different structures. Different antibody regions can be illustrated by reference to IgG. An IgG molecule contains four polypeptide chains, two longer length heavy chains and two shorter light chains that are inter-connected by disulfide bonds. The heavy and light chains each contain a constant region and a variable region. A heavy chain is comprised of a heavy chain variable region (VH) and a heavy chain constant region (CH1, CH2 and CH3). A light chain is comprised of a light chain variable region (VL) and a light chain constant region (CL). There are three hypervariable regions within the variable regions that are responsible for antigen specificity.

The hypervariable regions are generally referred to as complementarity determining regions (“CDR”) and are interposed between more conserved flanking regions referred to as framework regions (“FW”). There are four (4) FW regions and three (3) CDRs that are arranged from the N12 terminus to the COOH terminus as follows: FW1, CDR1, FW2, CDR2, FW3, CDR3, FW4. For example, the framework regions and CDRs can be identified from consideration of both the Kabat and Chothia definitions. The variable regions of the heavy and light chains contain a binding domain that interacts with an antigen. The two heavy chain carboxyl regions are constant regions joined by disulfide bonding to produce an Fc region. The Fc region is important for providing effector functions. Each of the two heavy chains making up the Fc region extends into different Fab regions through a hinge region.

The anti-PD-1 agents or the anti-CTLA-4 agents typically contain an antibody variable region. Such antibody fragments include but are not limited to (i) a Fab fragment, a monovalent fragment consisting of the VH, VL, CH and CL domains; (ii) a Fab2 fragment, a bivalent fragment comprising two Fab fragments linked by a disulfide bridge at the hinge region; (iii) a Fd fragment consisting of the VH, and CH1 domains; (iv) a Fv fragment consisting of the VH and VL domains of a single arm of an antibody; (v) a dAb fragment, which comprises either a VH or VL domain; (vi) a scAb, an antibody fragment containing VH and VL as well as either C1 or CH1 and (vii) artificial antibodies based upon protein scaffolds, including but not limited to fibronectin type III polypeptide antibodies. Furthermore, although the two domains of the Fv fragment, VL and VH, are coded for by separate genes, they can be joined using recombinant methods by a synthetic linker that enables them to be made as a single protein chain in which the VL and VH regions pair to form monovalent molecules, known as single chain Fv (scFv). Thus, the antibody variable region can be present in a recombinant derivative. Examples of recombinant derivatives include single-chain antibodies, diabody, triabody, tetrabody, and miniantibody. An anti-PD-1 agent or an anti-CTLA-4 agent can also contain one or more variable regions recognizing the same or different epitopes.

In some embodiments, anti-PD-1 agents or anti-CTLA-4 agents are encoded by an oncolytic virus produced using recombinant nucleic acid techniques. Different anti-PD-1 agents can be produced by different techniques, including, for example, a single chain protein containing a VH region and VL region connected by a linker sequence, such as a scFv, and antibodies or fragments thereof, and a multi-chain protein containing a VH and VL region on separate polypeptides. Recombinant nucleic acid techniques involve constructing a nucleic acid template for protein synthesis. Suitable recombinant nucleic acid techniques are well known in the art. Recombinant nucleic acid encoding an anti-PD-1 antibody or an anti-CTLA-4 antibody can be expressed in a cell that has been infected with an oncolytic virus and released into the tumor microenvironment upon viral lysis. The cell in effect serves as a factory for the encoded protein.

A nucleic acid comprising one or more recombinant genes encoding for either or both of an anti-PD-1 or anti-CTLA-4 agent VH region or VL region can be used to produce a complete protein/polypeptide binding to PD-1/CTLA-4. A complete binding agent can be provided, for example, using a single gene to encode a single chain protein containing a VH region and VL region connected by a linker, such as a scFv, or using multiple recombinant regions to, for example, produce both VH and VL regions.

Exemplary anti-PD-1 antibodies or anti-CTLA-4 antibodies, or its fragments or derivatives useful for the present disclosure are available in the art. See for example WO 2006/121168, WO 2014/055648, WO 2008/156712, US 2014/0234296, or U.S. Pat. No. 6,984,720.

### Pharmaceutical Compositions

In another aspect, the present disclosure provides a pharmaceutical composition for treatment of a tumor, comprising an effective amount of the genetically engineered oHSV-1 as described herein and a pharmaceutically acceptable carrier.

In some embodiments, a pharmaceutical composition for treatment of a tumor, comprises an effective amount of a genetically engineered oHSV-1, and a pharmaceutically acceptable carrier, wherein the genetically engineered oHSV-1 comprises a modified genome, wherein the modification comprises (a) an alternation of a copy of γ34.5 gene that is in a terminal repeat of the genome, rendering that copy of γ34.5 gene incapable of expressing functional ICP34.5 protein, and (b) a deletion of an internal inverted repeat region of the genome, causing one copy of each of double-copy genes and one copy of duplicated non-coding sequences within the internal inverted repeat region deleted, wherein the double-copy genes comprise genes encoding ICP0, ICP4, ICP34.5, ORF P and ORF O, and wherein all single-copy genes in both U_{L} and U_{S} components of the genome are intact such that they are capable of expressing respective functional proteins.

In some embodiments, the alternation comprises a deletion of all or part of the coding or regulatory region of the copy of γ34.5 gene. In some embodiments, the duplicated non-coding sequences include introns of ICP0, LAT domain and “a” sequence. In some embodiments, the all single-copy genes in both U_{L} and U_{S} components include U_{L}1 to U_{L}56 genes in the U_{L} component and U_{S}1 to U_{S}12 genes in the U_{S} component.

In some embodiments, the oHSV-1 is selected from the group consisting of strains F, KOS, and 17. In some embodiments, the deletion of an internal inverted repeat region causes excision of nucleotide positions 117005 to 132096 in the genome of F strain.

In some embodiments, the oHSV-1 has a genome isomer of prototype (P) and the deletion of an internal inverted repeat region starts from the stop codon of the last gene (e.g. U_{L}56) in the U_{L} component to the promotor of the first gene (e.g. U_{S}1) in the U_{S} component.

In some embodiments, the oHSV-1 is incorporated with a heterologous nucleic acid sequence encoding an immunostimulatory and/or immunotherapeutic agent, wherein the incorporation does not interfere with the expression of native genes of the HSV-1 genome. In some embodiments, the oHSV-1 is incorporated with a heterologous nucleic acid sequence encoding an immunostimulatory agent and an immunotherapeutic agent.

In some embodiments, the immunostimulatory agent is selected from a group consisting of GM-CSF, IL-2, IL-12, IL-15, IL-24 and IL-27. In some embodiments, the immunostimulatory agent is IL-12. In some embodiments, the immunotherapeutic agent is an anti-PD-1 agent, an anti-CTLA-4 agent or both. In some embodiments, the immunotherapeutic agent is an anti-PD-1 agent.

In some embodiments, the heterologous nucleic acid sequence is incorporated into the internal inverted repeat region and/or between U_{L}3 and U_{L}4 genes in the U_{L} component. In some embodiments, the oHSV-1 is incorporated with a heterologous nucleic acid sequence encoding IL-12 and an anti-PD-1 agent. In some embodiments, the heterologous nucleic acid sequence encoding IL-12 is incorporated into the internal inverted repeat region and the heterologous nucleic acid sequence encoding the anti-PD-1 agent is incorporated between U_{L}3 and U_{L}4 genes in the U_{L} component.

The oncolytic virus may be prepared in a suitable pharmaceutically acceptable carrier or excipient. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologies standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

In some embodiments, the composition disclosed herein is used for treatment of a tumor. In some embodiments, the composition disclosed herein is used for treatment of a solid tumor. In some embodiments, the composition disclosed herein is used for treatment of a brain tumor. In some embodiments, the composition disclosed herein is used for treatment of a brain tumor which is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

### Uses and Therapies

In another aspect, the present disclosure provides the genetically engineered oHSV-1 as described herein for use in treatment of a tumor in a subject. In another aspect, the present disclosure provides the genetically engineered oHSV-1 as described herein for use in treatment of a solid tumor in a subject. In another aspect, the present disclosure provides the genetically engineered oHSV-1 as described herein for use in treatment of a brain tumor in a subject.

In some embodiments, the genetically engineered oHSV-1 comprises a modified genome, wherein the modification comprises (a) an alternation of a copy of γ34.5 gene that is in a terminal repeat of the genome, rendering that copy of γ34.5 gene incapable of expressing functional ICP34.5 protein, and (b) a deletion of an internal inverted repeat region of the genome, causing one copy of each of double-copy genes and one copy of duplicated non-coding sequences within the internal inverted repeat region deleted, wherein the double-copy genes comprise genes encoding ICP0, ICP4, ICP34.5, ORF P and ORF O, and wherein all single-copy genes in both U_{L} and U_{S} components of the genome are intact such that they are capable of expressing respective functional proteins.

In some embodiments, the alternation comprises a deletion of all or part of the coding or regulatory region of the copy of γ34.5 gene. In some embodiments, the duplicated non-coding sequences include introns of ICP0, LAT domain and “a” sequence. In some embodiments, the all single-copy genes in both U_{L} and U_{S} components include U_{L}1 to U_{L}56 genes in the U_{L} component and U_{S}1 to U_{S}12 genes in the U_{S} component.

In some embodiments, the oHSV-1 is selected from the group consisting of strains F, KOS, and 17. In some embodiments, the deletion of an internal inverted repeat region causes excision of nucleotide positions 117005 to 132096 in the genome of F strain.

In some embodiments, the oHSV-1 has a genome isomer of prototype (P) and the deletion of an internal inverted repeat region starts from the stop codon of the last gene (e.g. U_{L}56) in the U_{L} component to the promotor of the first gene (e.g. U_{S}1) in the U_{S} component.

In some embodiments, the oHSV-1 is incorporated with a heterologous nucleic acid sequence encoding an immunostimulatory and/or immunotherapeutic agent, wherein the incorporation does not interfere with the expression of native genes of the HSV-1 genome. In some embodiments, the oHSV-1 is incorporated with a heterologous nucleic acid sequence encoding an immunostimulatory agent and an immunotherapeutic agent.

In some embodiments, the immunostimulatory agent is selected from a group consisting of GM-CSF, IL-2, IL-12, IL-15, IL-24 and IL-27. In some embodiments, the immunostimulatory agent is IL-12. In some embodiments, the immunotherapeutic agent is an anti-PD-1 agent, an anti-CTLA-4 agent or both. In some embodiments, the immunotherapeutic agent is an anti-PD-1 agent.

In some embodiments, the heterologous nucleic acid sequence is incorporated into the internal inverted repeat region and/or between U_{L}3 and U_{L}4 genes in the U_{L} component. In some embodiments, the oHSV-1 is incorporated with a heterologous nucleic acid sequence encoding IL-12 and an anti-PD-1 agent. In some embodiments, the heterologous nucleic acid sequence encoding IL-12 is incorporated into the internal inverted repeat region and the heterologous nucleic acid sequence encoding the anti-PD-1 agent is incorporated between U_{L}3 and U_{L}4 genes in the U_{L} component.

In another aspect, the present disclosure provides use of the genetically engineered oHSV-1 as described herein in the manufacturing of a drug for treatment of a tumor in a subject. In another aspect, the present disclosure provides use of the genetically engineered oHSV-1 as described herein in the manufacturing of a drug for treatment of a solid tumor in a subject. In another aspect, the present disclosure provides use of the genetically engineered oHSV-1 as described herein in the manufacturing of a drug for treatment of a brain tumor in a subject.

In some embodiments, the genetically engineered oHSV-1 comprises a modified genome, wherein the modification comprises (a) an alternation of a copy of γ34.5 gene that is in a terminal repeat of the genome, rendering that copy of γ34.5 gene incapable of expressing functional ICP34.5 protein, and (b) a deletion of an internal inverted repeat region of the genome, causing one copy of each of double-copy genes and one copy of duplicated non-coding sequences within the internal inverted repeat region deleted, wherein the double-copy genes comprise genes encoding ICP0, ICP4, ICP34.5, ORF P and ORF O, and wherein all single-copy genes in both U_{L} and U_{S} components of the genome are intact such that they are capable of expressing respective functional proteins.

In some embodiments, the alternation comprises a deletion of all or part of the coding or regulatory region of the copy of γ34.5 gene. In some embodiments, the duplicated non-coding sequences include introns of ICP0, LAT domain and “a” sequence. In some embodiments, the all single-copy genes in both U_{L} and U_{S} components include U_{L}1 to U_{L}56 genes in the U_{L} component and U_{S}1 to U_{S}12 genes in the U_{S} component.

In some embodiments, the oHSV-1 is selected from the group consisting of strains F, KOS, and 17. In some embodiments, the deletion of an internal inverted repeat region causes excision of nucleotide positions 117005 to 132096 in the genome of F strain.

In some embodiments, the oHSV-1 has a genome isomer of prototype (P) and the deletion of an internal inverted repeat region starts from the stop codon of the last gene (e.g. U_{L}56) in the U_{L} component to the promotor of the first gene (e.g. U_{S}1) in the U_{S} component.

In some embodiments, the oHSV-1 is incorporated with a heterologous nucleic acid sequence encoding an immunostimulatory and/or immunotherapeutic agent, wherein the incorporation does not interfere with the expression of native genes of the HSV-1 genome. In some embodiments, the oHSV-1 is incorporated with a heterologous nucleic acid sequence encoding an immunostimulatory agent and an immunotherapeutic agent.

In some embodiments, the immunostimulatory agent is selected from a group consisting of GM-CSF, IL-2, IL-12, IL-15, IL-24 and IL-27. In some embodiments, the immunostimulatory agent is IL-12. In some embodiments, the immunotherapeutic agent is an anti-PD-1 agent, an anti-CTLA-4 agent or both. In some embodiments, the immunotherapeutic agent is an anti-PD-1 agent.

In some embodiments, the heterologous nucleic acid sequence is incorporated into the internal inverted repeat region and/or between U_{L}3 and U_{L}4 genes in the U_{L} component. In some embodiments, the oHSV-1 is incorporated with a heterologous nucleic acid sequence encoding IL-12 and an anti-PD-1 agent. In some embodiments, the heterologous nucleic acid sequence encoding IL-12 is incorporated into the internal inverted repeat region and the heterologous nucleic acid sequence encoding the anti-PD-1 agent is incorporated between U_{L}3 and U_{L}4 genes in the U_{L} component.

In another aspect, the present disclosure provides a method for treating or alleviating a tumor in a subject, comprising administering to a subject in need thereof an effective amount of the oHSV-1 virus or the pharmaceutical composition comprising the oHSV-1 virus as described herein. In certain embodiments, the tumor is a solid tumor. In certain embodiments, the tumor is a brain tumor. In some embodiments, the brain tumor is selected from a group consisting of glioma, glioblastoma, oligodendroglioma, astrocytoma, ependymoma, primitive neuroectodermal tumor, atypical meningioma, malignant meningioma, and neuroblastoma. In some embodiments, the brain tumor is glioblastoma multiform.

In certain embodiments, the oHSV-1 virus or the pharmaceutical composition is administered intratumorally. In an embodiment, the HSV-1 virus or the pharmaceutical composition is injected directly to a tumor mass in the form of an injectable solution.

The methods of the invention are useful for treating brain tumors. This includes all tumors inside the human skull (cranium) or in the central spinal canal. The tumor may originate from the brain itself, but also from lymphatic tissue, blood vessels, the cranial nerves, the brain envelopes (meninges), skull, pituitary gland, or pineal gland. Within the brain itself, the involved cells may be neurons or glial cells (which include astrocytes, oligodendrocytes, and ependymal cells). Brain tumors may also spread from cancers primarily located in other organs (metastatic tumors).

In some embodiments, the brain tumor is a glioma, such as an ependymoma, astrocytoma, oligoastrocytoma, oligodendroglioma, ganglioglioma, glioblastoma (also known as glioblastoma multiforme), or mixed glioma. Gliomas are primary brain tumors and are classified into four grades (I, II, III, and IV) based on their appearance under a microscope, and particularly the presence of atypical cells, mitoses, endothelial proliferation, and necrosis. Grade I and II tumors, termed “low-grade gliomas,” have none or one of these features and include diffuse astrocytomas, pilocytic astrocytomas, low-grade astrocytomas, low-grade oligoastrocytomas, low-grade oligodendrogliomas, gangliogliomas, dysembryoplastic neuroepithelial tumors, pleomorphic xanthoastrocytomas, and mixed gliomas. Grade III and IV tumors, termed “high-grade gliomas,” have two or more of these features and include anaplastic astrocytomas, anaplastic oligodendrogliomas, anaplastic oligoastrocytomas, anaplastic ependymomas, and glioblastomas (including giant cell glioblastomas and gliosarcomas). In one aspect of these embodiments, the glioma is a low-grade glioma. In another aspect of these embodiments, the glioma is a high-grade glioma. In another aspect of these embodiments, the glioma is a glioblastoma.

In some embodiments, it may be desirable to combine the oHSV-1 with other agents effective in the treatment of cancer. For example, the treatment of a cancer may be implemented with an oncolytic virus and other anti-cancer therapies, such as anti-cancer agents or surgery. In the context of the present technology, it is contemplated that oncolytic virus therapy could be used in conjunction with chemotherapeutic, radiotherapeutic, immunotherapeutic or other biological intervention.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In some embodiments, the oHSV-1 disclosed herein is combined with an adjuvant. In one embodiment, the adjuvant is an oligonucleotide comprising an unmethylated CpG motif. Unmethylated dinucleotide CpG motifs in bacterial deoxyribonucleic acid (DNA) have advantages for stimulating several immune cells to secrete cytokines for enhancements of innate and adaptive immunity.

The viral therapy may precede or follow the other agent treatment by intervals ranging from minutes to weeks. In embodiments where the other agent and oncolytic virus are applied separately to the cell, one would generally ensure that a significant period of time did not expire between the time of each delivery, such that the agent and virus would still be able to exert an advantageously combined effect on the cell. In such instances, it is contemplated that one may contact the cell with both modalities within about 12-24 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In some embodiments, a second therapy is administered to the subject before, at the same time, or after the oHSV-1 disclosed herein or the pharmaceutical composition disclosed herein is administered. In some embodiments, the second therapy is chemotherapeutic, radiotherapeutic, immunotherapeutic and/or surgery intervention. In some embodiments, the subject is a human being.

Sequences used in the present disclosure is summarized below.

| SEQ | |
|---|---|
| ID | |
| NO: | Nucleic Acid or Amino Acid |
| # | Sequences |
| 1 | EVQLVESGGGVVQPGRSLRLSCAASGFTFSSYLMSWVRQAPGKG |
| | LEWVATISGGGGDTYFPDSVKGRFTISRDNSKNTLYLQMNSLRAE |
| | DTAVYYCVRFGGAGYYWYFDVWGQGTLVTVSS (anti-hPD-1 |
| | Fab heavy chain variable region) |
| 2 | ASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPS |
| | NTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYN |
| | STYRVVSVLTVLHQDWLNGKEYKCKVSNKALPAPIEKTISKAKG |
| | QPREPQVYTLPPSREEMTKNQVSLTCLVKGFYPSDIAVEWESNGQ |
| | PENNYKTTPPVLDSDGSFFLYSKLTVDKSRWQQGNVFSCSVMHE |
| | ALHNHYTQKSLSLSPGK (anti-hPD-1 Fab heavy |
| | chain constant region) |
| 3 | EIVLTQSPATLSLSPGERATLSCRASKSVDDSGISFMHWYQQKPG |
| | QAPRLLIYAASNQGSGIPARFSGSGSGTDFTLTISSLEPEDFAV |
| | YYCHQTKEVPWTFGQGTKVEIK (anti-hPD-1 Fab light |
| | chain variable region) |
| 4 | RTVAAPSVFIFPPSDEQLKSGTASVVCLLNNFYPREAKVQWKVDN |
| | ALQSGNSQESVTEQDSKDSTYSLSSTLTLSKADYEKHKVYACEVT |
| | HQGLSSPVTKSFNRGEC (anti-hPD-1 Fab light |
| | chain constant region) |
| 5 | GAAGATCTAATATTTTTATTGCAACTCCCTG (primer) |
| 6 | CTAGCTAGCTTATAAAAGGCGCGTCCCGTGG (primer) |
| 7 | GCTCTAGATTGCGACGCCCCGGCTC (primer) |
| 8 | CCTTAATTAAGGTTACCACCCTGTAGCCCCGATGT (primer) |
| 9 | TCCCATGGATTTAACAAACGGGGGGGTGTCG (primer) |
| 10 | GGCCCCCGAGGCCAGCATGACGTTATCT (primer) |
| 11 | GAGTAACCGCCCCCCCCCCATGCCACCCTCAC (primer) |
| 12 | GTGTTTTACTGCCACTACACCCCCGGGGAAC (primer) |
| 13 | EVMLVESGGGLVKPGGSLKLSCAASGFTFSSYLMSWVRQTPEKR |
| | LEWVATISGGGGDTYFPDSVKGRFTISRDNVKNNLYLQMSSLRSE |
| | DTALYYCVRFGGAGYYWYFDVWGAGTTVTVSS (anti-mPD-1 |
| | scFv heavy chain variable region) |
| 14 | DIVLTQSPASLAVSLGQRATISCRASKSVDDSGISFMHWFQQKPG |
| | QPPKLLIYAASNQGSGVPARFRGSGSGTDFSLNIHPMEEDDTAMY |
| | FCHQTKEVPWTFGGGTKLEIK (anti-mPD-1 scFv light |
| | chain variable region) |

### Examples

As demonstrated in the examples here below, a genetically engineered oHSV-1 virus with both copies of γ34.5 gene deleted and a further deletion of the internal inverted repeat region exhibited surprisingly and unexpected higher anti-tumor activity on various brain tumor cells over non-brain tumor cells or normal cells. These results are surprising in view that oHSV-1 viruses with similar genome structures as known in the art (such as T3011, R3616, WT strain F) is less efficient in brain tumor cell killing than the oHSV-1 disclosed herein (i.e., C1212, C5252, C8282).

Constructions of oHSV-1 C5252, C8282, and C1212
Construction of oHSV-1 C5252
C5252 comprises the deletion of γ34.5 genes, an insertion of an anti-human PD-1 antibody expression cassette between U_{L}3 and U_{L}4, and a modified internal repeat (IR) region replaced by an IL-12 expression cassette. The recombinant virus was constructed in several steps with the aid of bacterial artificial chromosome (BAC) system. The details of viral construction are described following.

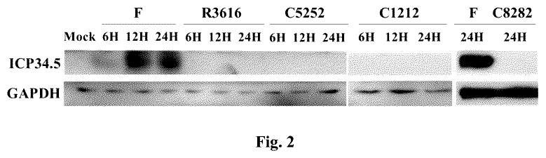

The HSV-1 BAC with two copies of γ34.5 genes deletion (BAC-Δ34.5) arrangement is used. IL-12 expression cassettes flanked by upstream of nucleotides 117005 and downstream of nucleotides 132096 in the context of a wild type genome were PCR amplified from HSV-1 viral genome by two sets of primers respectively (GAAGATCTAATATTTTTATTGCAACTCCCTG (SEQ ID NO: 5), CTAGCTAGCTTATAAAAGGCGCGTCCCGTGG (SEQ ID NO: 6)) and (GCTCTAGATTGCGACGCCCCGGCTC (SEQ ID NO: 7), CCTTAATTAAGGTTACCACCCTGTAGCCCCGATGT (SEQ ID NO: 8)) and inserted into a gene replacement plasmid pKO5 to generate pKO1407. pKO1407 was then transfected to Escherichia coli with BAC-Δ34.5 by electroporation to generate BAC-Δ34.5-IL12. Then, cassette of CMV promoter driving the PD-1 Fab gene flanked by upstream of nucleotides 11658 and downstream of nucleotides 11659 in the context of a wild type genome were PCR amplified from HSV-1 viral genome by two sets of primers respectively (TCCCATGGATTTAACAAACGGGGGGGTGTCG (SEQ ID NO: 9), GGCCCCCGAGGCCAGCATGACGTTATCT (SEQ ID NO: 10)) and (GAGTAACCGCCCCCCCCCCATGCCACCCTCAC (SEQ ID NO: 11), GTGTTTTACTGCCACTACACCCCCGGGGAAC (SEQ ID NO: 12)) and ligated into pKO5 at the sites of BglII and PacI to generate the pKOE1002 plasmid. pKOE1002 plasmid was then transfected to Escherichia coli harboring BAC-Δ34.5-IL12 by electroporation to generate BAC-5252. C5252 virus was obtained by transfection of BAC-5252 plasmid following by several step plaque purification and amplification in Vero cells followed by identification of virus by detection of IL-12 and PD-1 Fab secretion (Table 1) and γ34.5 gene coding protein ICP34.5 expression (FIG. 2).

C8282 is a functionally identical mouse version of C5252 except that C8282 carries a mouse version of IL-12 and a mouse anti-PD-1 antibody (single chain antibody fragment, scFv, containing heavy chain variable region and light chain variable region having the sequences set forth in SEQ ID NOs: 13 and 14, respectively) in the same location on the viral genome where C5252 carries a human IL-12 and an anti-human PD-1 antibody.

C1212 is a functionally identical version of C5252 except that C1212 carries a CMV promoter followed by three repeat stop codon and green fluorescent protein (GFP) in the same location on the viral genome where C5252 carries a human IL-12 and an anti-human PD-1 antibody (PD-1 Fab, containing heavy chain variable region and constant region as well as light chain variable region and constant region having the sequences set forth in SEQ ID NOs: 1-4, respectively).

### Confirmation of IL-12 and Anti-PD-1 Antibody Expression and ICP34.5 Protein Expression by C5252, C8282, and C1212 Virus

Vero cells were seeded into 6-well plates at a density of 4×10⁵ cells per well. After overnight incubation, the cells were mock infected or infected at 1 PFU of HSV-1 (F), R3616, C5252, C8282, C1212 per cell. The cells were harvested at 6, 12 and 24 hours (H) post infection respectively. The proteins were electrophoretically separated in 10% denaturing gels and reacted with antibodies ICP34.5 or GAPDH. GAPDH was served as loading control (FIG. 2). The cell supernatants collected at 24-hour (H) post-infection of C5252, C8282, and C1212 were used for ELISA assay to detect the expression levels of IL-12 and anti-PD-1 antibody. The results were shown in Table 1.

TABLE 1
Detection of IL-12 and anti-PD-1 antibody (PD-1 Ab)
expression by C5252, C8282, and C1212.

| oHSV | IL-12 (pg/mL) | PD-1 Ab (pg/mL) |
|---|---|---|
| C5252 | 762.8 | 377.58 |
| C8282 | 724.09 | 519.16 |
| C1212 | U | U |

*U: Undetectable

As shown in Table 1, IL-12 and anti-PD-1 antibody were detected at comparable levels expressed by C5252 and C8282 virus. C1212 which is a backbone virus was undetectable of IL-12 as well as anti-PD-1 antibody expression determined by ELISA assay.

ICP34.5 protein expression detected by immunoblotting as shown in FIG. 2 indicated that ICP34.5 protein was lacking expression in C5252, C8282, C1212 and R3616 infection samples, while was expressed in wild type (WT) F infection samples.

All of above results demonstrated that the recombinant viruses C5252, C8282, and C1212 were confirmed by IL-12, anti-PD-1 antibody expression and absence of ICP34.5 protein expression.

### In Vitro Cell Killing Activity—Brain Tumor Cell Lines

Δ172, D54-MG, U87-MG, U138-MG and D458 cells were seeded onto 96-well plate (4000 cells/well) and infected with F, R3616, T3011 and C5252 (0.1 and 1.0 PFU/Cell). After 48 hours infection (48H p.i.), the cell viability was determined by CCK8-Kit. Inhibition Rate=(OD of the uninfected well−OD of the oHSV infected well)/(OD of the uninfected well−OD of the blank well)×100%. The blank well containing culture medium only. All values in the experiments were expressed as mean±SEM. Results were shown in Table 2.

TABLE 2
In vitro cell killing activities of HSV-1 WT F, R3616, T3011
and C5252 on brain tumor cell lines

| | | Inhibition Rate ± SEM (%) (48 H p.i.) | |
|---|---|---|---|
| Cell Line | Virus | 0.1 PFU/Cell | 1.0 PFU/Cell |
| A172 | HSV-1(F) | 36.23 ± 18.30 | 63.62 ± 7.01 |
| | T3011 | 10.26 ± 15.80 | 58.11 ± 4.32 |
| | R3616 | 3.80 ± 6.64 | 57.52 ± 4.31 |
| | C5252 | 33.70 ± 7.04 | 77.59 ± 6.89 |
| D54-MG | HSV-1(F) | 9.25 ± 1.56 | 18.91 ± 6.59 |
| | T3011 | 0 | 0.67 ± 5.96 |
| | R3616 | 0 | 0 |
| | C5252 | 0 | 28.23 ± 7.06 |
| U87-MG | HSV-1(F) | 0 | 82.03 ± 0.09 |
| | T3011 | 19.78 ± 0.61 | 63.20 ± 3.08 |
| | R3616 | 0 | 30.35 ± 2.74 |
| | C5252 | 19.37 ± 0.58 | 59.21 ± 1.25 |
| U138-MG | HSV-1(F) | 19.42 ± 6.74 | 49.71 ± 3.02 |
| | T3011 | 0 | 0 |
| | R3616 | 0 | 19.23 ± 9.70 |
| | C5252 | 0 | 53.49 ± 4.02 |
| D458 | HSV-1(F) | 0 | 81.99 ± 0.11 |
| | T3011 | 19.78 ± 0.61 | 60.03 ± 3.26 |
| | R3616 | 0 | 25.10 ± 1.18 |
| | C5252 | 19.37 ± 0.58 | 56.89 ± 0.66 |

As shown in Table 2, oHSV-1 C5252 was an effective cell killing agent in all of the tumor brain cells tested at 1.0 PFU/Cell. In cell lines Δ172, D54-MG, U138-MG, C5252 represented the highest cell killing ability among the oHSV-1 viruses tested. For cell lines U87-MG and D458, the anti-tumor effect of C5252 was comparable to T3011. Compared to R3616 which is also γ34.5 gene null oHSV-1, C5252 was almost 2- to 3-fold effective in most of the cell lines tested.

### In Vitro Cell Killing Activity—Non-Brain Tumor Cell Lines

Cells were seeded onto 96-well plate (4000 cells/well) and infected with F, T3011 and C5252 (0.1 and 1.0 PFU/Cell). After 48 hours infection (48H p.i.), the cell viability was determined by CCK8-Kit. Inhibition Rate=(GD of the uninfected well-GD of the oHSV infected well)/(GD of the uninfected well−GD of the blank well)×10000. The blank well containing culture medium only. All values in the experiments were expressed as mean±SEM. Results were shown in Table 3.

TABLE 3
In vitro cell killing activities of HSV-1 WT F, R3616, T3011
and C5252 on non-brain tumor cell lines

| | | Inhibition Rate ± SEM (%) (48 H p.i.) | |
|---|---|---|---|
| Cell Line | Virus | 0.1 PFU/Cell | 1.0 PFU/Cell |
| HEp-2 | HSV-1(F) | 10.49 ± 6.66 | 43.38 ± 4.78 |
| | T3011 | 10.39 ± 11.06 | 28.20 ± 5.02 |
| | C5252 | 9.73 ± 5.55 | 26.02 ± 2.92 |
| CNE1 | HSV-1(F) | 8.20 ± 6.18 | 57.23 ± 0.53 |
| | T3011 | 9.99 ± 9.47 | 45.78 ± 4.11 |
| | C5252 | 5.48 ± 0.75 | 48.14 ± 6.51 |
| HLAMP | HSV-1(F) | 0 | 46.89 ± 1.26 |
| | T3011 | 0 | 51.79 ± 4.38 |
| | C5252 | 0 | 49.39 ± 8.05 |
| MDA-MB-231 | HSV-1(F) | 47.21 ± 3.35 | 64.19 ± 4.86 |
| | T3011 | 49.58 ± 9.08 | 71.13 ± 2.44 |
| | C5252 | 22.26 ± 10.03 | 63.75 ± 3.02 |
| SCC25 | HSV-1(F) | 58.03 ± 6.56 | 81.99 ± 1.87 |
| | T3011 | 37.53 ± 4.78 | 80.52 ± 3.15 |
| | C5252 | 23.43 ± 6.22 | 79.05 ± 1.87 |
| KYSE30 | HSV-1(F) | 24.87 ± 6.00 | 64.75 ± 3.94 |
| | T3011 | 17.15 ± 1.67 | 66.04 ± 5.48 |
| | C5252 | 15.87 ± 13.57 | 46.05 ± 3.83 |
| 5637 | HSV-1(F) | 32.47 ± 18.97 | 69.19 ± 7.16 |
| | T3011 | 29.56 ± 6.81 | 75.41 ± 6.24 |
| | C5252 | 21.27 ± 8.51 | 79.88 ± 2.59 |
| HCT116 | HSV-1(F) | 7.56 ± 7.20 | 56.67 ± 6.25 |
| | T3011 | 0 | 47.67 ± 3.07 |
| | C5252 | 0 | 41.90 ± 5.08 |

As shown in Table 3, when tested in non-brain tumor cell lines, both T3011 and C5252 were effective tumor killing agents against various non-brain tumor cells. It was noted that the anti-tumor activity of C5252 is substantially equivalent to T3011 in all of the 8 cell lines tested in this example, either at a lower or a higher multiplicity of infection (MOI). This was not expected because C5252 is a further attenuated version of T3011 with the second copy of the γ34.5 gene deleted. The deletion of the second copy of the γ34.5 gene, however, showed no adverse effect to the anti-tumor activity against non-brain tumor cells of the oHSV-1 virus, but significantly improved its tumor killing effect against brain tumor cells as shown in Table 2. The oHSV-1 virus as disclosed herein is thus generally more effective in tumor killing than the oHSV-1 from which it derived.

### In Vitro Inhibitory Assessment of C5252 on Proliferation of Human Malignant Glioma Cells

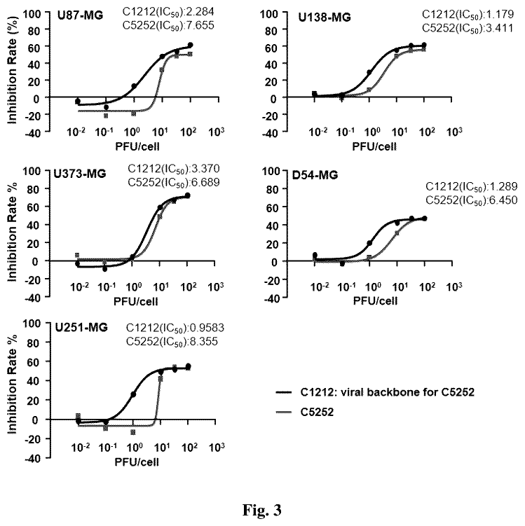

As shown in FIG. 3, the sensitivity of C5252 to human glioma cell lines U87-MG, U138-MG, U373-MG, D54-MG and U251-MG were basically the same, and the IC₅₀ value for C5252 and C1212 against these glioma cells were less than 10 MOI. The inhibitory effect for C5252 is comparable to backbone C1212. The incorporation of heterologous genes into the genome of the virus did not substantially impact the replication and thus the inhibitory capability of the oHSV, but when administered in vivo would significantly aid in tumor cell killing by immune system of the subject due to the nature of the immunostimulatory (IL-12) and immunotherapeutic agents (anti-PD-1 antibody) expressed by the oHSV-1 virus.

### The Inhibitory Effect of C5252 on Normal Cells and Tumor Cells

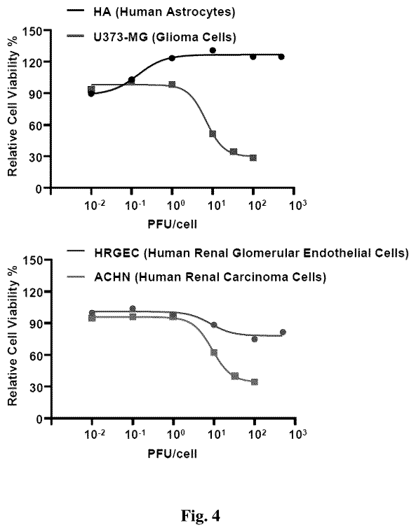

As shown in FIG. 4, the IC₅₀ values of C5252 on tumor cells U373-MG and ACHN were 6.890 and 9.102 MOI, respectively, and the IC₅₀ values of C5252 on normal cells HA and HRGEC were all greater than 500 MOI. Under the conditions of this experiment, C5252 showed no obvious inhibitory effect on normal cells, but showed a significant inhibitory effect on tumor cells. Compared with normal cells, the inhibitory effect of C5252 had a higher targeting effect on human tumor cells. The results indicated that C5252 selectively kills tumor cells while sparing normal cells.

### Efficacy Study of C8282 in the Treatment of GL261 Subcutaneously Implanted Model in C57BL/6 Mice

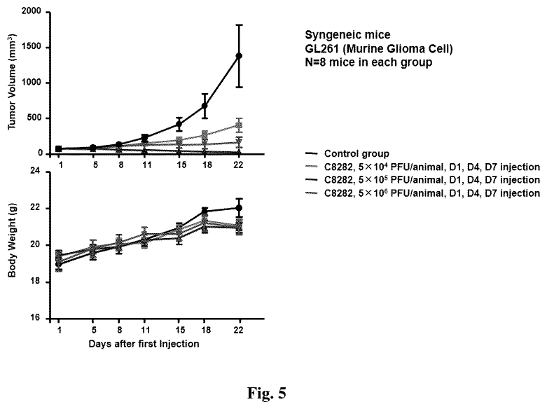

C8282 is a mouse surrogate for C5252, in which mouse IL-12 (m-IL-12) and anti-mouse PD-1 (m-PD-1) antibody were introduced into the virus genome to replace respective human counterparts. As shown in FIG. 5, C8282 intra-tumoral injection showed significant efficacy against GL261 subcutaneously tumor model. Animals were well tolerated when mice were treated with C8282 at dose≤5×10⁶ PFU/animal. Medium dosage level at 5×10⁵ PFU/animal appeared exhibiting highest efficacy among the tested dosage ranges.

### Efficacy Study of C5252 in the Treatment of Orthotropic U87 Human Glioma Model in Nude Mice

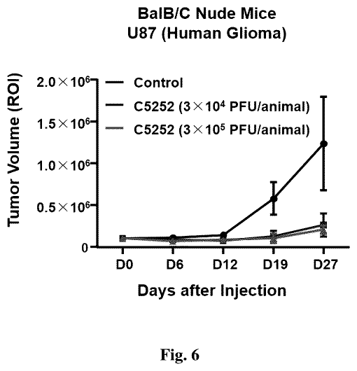

As shown in FIG. 6, C5252 intracerebral injection showed significant efficacy against U87-MG cell in nude mice. Different dosage levels did not show significant difference.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

1. An oncolytic Herpes Simplex Virus type 1 (oHSV-1) comprising a modified genome, wherein the modification comprises
a) an alternation of a copy of γ34.5 gene that is in a terminal repeat of the genome, rendering that copy of γ34.5 gene incapable of expressing functional ICP34.5 protein, and
b) a deletion of an internal inverted repeat region of the genome, causing one copy of each of double-copy genes and one copy of duplicated non-coding sequences within the internal inverted repeat region deleted,
wherein the double-copy genes comprise genes encoding ICP0, ICP4, ICP34.5, ORF P and ORF O, and
wherein all single-copy genes in both U_{L} and U_{S} components of the genome are intact such that they are capable of expressing respective functional proteins.

2. The oHSV-1 of claim 1, wherein the alternation comprises a deletion of all or part of the coding or regulatory region of the copy of γ34.5 gene.

3. The oHSV-1 of claim 1, wherein the duplicated non-coding sequences include introns of ICP0, LAT domain and “a” sequence.

4. The oHSV-1 of claim 1, wherein the all single-copy genes in both U_{L} and U_{S} components include U_{L}1 to U_{L}56 genes in the U_{L} component and U_{S}1 to U_{S}12 genes in the U_{S} component.

5. The oHSV-1 of claim 1, wherein the HSV-1 is selected from the group consisting of strains F, KOS, and 17.

6. The oHSV-1 of claim 1, wherein the HSV-1 has a genome isomer of prototype (P).

7. The oHSV-1 of claim 6, wherein the deletion of an internal inverted repeat region causes excision of nucleotide positions 117005 to 132096 in the genome of F strain.

8. The oHSV-1 of claim 6, wherein the deletion of an internal inverted repeat region starts from the stop codon of the last gene in the U_{L} component to the promotor of the first gene in the U_{S} component.

9. The oHSV-1 of claim 8, wherein the last gene in the U_{L} component is U_{L}56 gene.

10. The oHSV-1 of claim 8, wherein the first gene in the U_{S} component is U_{S}1 gene.

11. The oHSV-1 of claim 1, wherein the oHSV-1 is incorporated with a heterologous nucleic acid sequence encoding an immunostimulatory and/or immunotherapeutic agent, wherein the incorporation does not interfere with the expression of native genes of the HSV-1 genome.

12. The oHSV-1 of claim 11, wherein the oHSV-1 is incorporated with a heterologous nucleic acid sequence encoding an immunostimulatory agent and an immunotherapeutic agent.

13. The oHSV-1 of claim 11, wherein the immunostimulatory agent is selected from a group consisting of GM-CSF, IL-2, IL-12, IL-15, IL-24 and IL-27.

14. The oHSV-1 of claim 13, wherein the immunostimulatory agent is IL-12.

15. The oHSV-1 of claim 11, wherein the immunotherapeutic agent is an anti-PD-1 agent, an anti-CTLA-4 agent or both.

16. The oHSV-1 of claim 15, wherein the immunotherapeutic agent is an anti-PD-1 agent.

17. The oHSV-1 of claim 11, wherein the heterologous nucleic acid sequence is incorporated into the internal inverted repeat region and/or between U_{L}3 and U_{L}4 genes in the U_{L} component.

18. The oHSV-1 of claim 11, wherein the oHSV-1 is incorporated with a heterologous nucleic acid sequence encoding IL-12 and an anti-PD-1 agent.

19. The oHSV-1 of claim 18, wherein the heterologous nucleic acid sequence encoding IL-12 is incorporated into the internal inverted repeat region and the heterologous nucleic acid sequence encoding the anti-PD-1 agent is incorporated between U_{L}3 and U_{L}4 genes in the U_{L} component.

20. A pharmaceutical composition, comprising an effective amount of the oHSV-1 of claim 1 and a pharmaceutically acceptable carrier.

21-27. (canceled)

28. A method for treating or alleviating a tumor in a subject, comprising administering to the subject in need thereof an effective amount of the oHSV-1 of claim 1, or a pharmaceutical composition comprising the oHSV-1.

29. The method of claim 28, wherein the tumor is a brain tumor.

30. The method of claim 29, wherein the brain tumor is selected from a group consisting of glioma, glioblastoma, oligodendroglioma, astrocytoma, ependymoma, primitive neuroectodermal tumor, atypical meningioma, malignant meningioma, and neuroblastoma.

31. The method of claim 30, wherein the brain tumor is glioblastoma multiform.
